(19) United States
(12) Patent Application Publication
(10) Pub. No.: US 2005/0187615 A1
(43) Pub. Date: Aug. 25, 2005

(54) Polymeric endoprostheses with enhanced strength and flexibility and methods of manufacture

(76) Inventors: Michael S. Williams, Santa Rosa, CA (US); Kevin D. Holbrook, Chapel Hill, NC (US); Richard A. Glenn, Santa Rosa, CA (US)

Correspondence Address: DEANNA J. SHIRLEY, 3418 BALDWIN WAY, SANTA ROSA, CA, 95403, US

(21) Appl. No.: 11/062,160

(22) Filed: Feb. 18, 2005

Related U.S. Application Data
(60) Provisional application No. 60/546,905, filed on Feb. 23, 2004.

(51) Int. Cl. A61F 2/06; A61F 2/04

(52) U.S. Cl. 623/1.34; 623/1.49; 623/23.71; 264/210.2; 264/151; 264/235

(57) ABSTRACT

Improved polymeric endoprostheses and methods of manufacturing endoprostheses are disclosed herein. The endoprostheses may comprise one or more polymers wherein the polymer chains are substantially aligned circumferentially, and comprising increased radial strength and flexibility. An endoprosthesis according to the invention may comprise a smooth surface. Endoprostheses disclosed herein may be used in the treatment of strictures in lumens of the body. Alternatively, endoprostheses disclosed herein may be used as anchors to secure medical devices within lumens of the body. The endoprostheses disclosed herein may comprise one or more erodible polymer.

### RELATED APPLICATIONS

This application is related to and claims the benefit of the priority date of U.S. Provisional Patent Application Ser. No. 60/546,905 entitled “Polymeric Endo-prostheses with Enhanced Strength and Flexibility and Methods of Manufacture”, filed Feb. 23, 2004, by Williams, et al.

### FIELD OF THE INVENTION

The invention herein relates generally to medical devices and the manufacture thereof, and to improved methods for manufacturing endoprostheses. Endoprostheses disclosed herein may be for use in the treatment of strictures in lumens of the body. Other embodiments disclosed herein may serve as anchors within lumens of the body for securing other medical devices. More particularly, the invention is directed to polymeric endoprostheses and addresses the shortcomings of the prior art, especially, but not limited to, material limitations such as radial strength and flexibility.

### BACKGROUND OF THE INVENTION

Ischemic heart disease is the major cause of death in industrialized countries. Ischemic heart disease, which often results in myocardial infarction, is a consequence of coronary atherosclerosis. Atherosclerosis is a complex chronic inflammatory disease and involves focal accumulation of lipids and inflammatory cells, smooth muscle cell proliferation and migration, and the synthesis of extracellular matrix. Nature 1993;362:801-809. These complex cellular processes result in the formation of atheromatous plaque, which consists of a lipid-rich core covered with a collagen-rich fibrous cap, varying widely in thickness. Further, plaque disruption is associated with varying degrees of internal hemorrhage and luminal thrombosis because the lipid core and exposed collagen are thrombogenic. J Am Coll Cardiol. 1994;23:1562-1569 Acute coronary syndrome usually occurs as a consequence of such disruption or ulceration of a so called “vulnerable plaque”. Arterioscler Thromb Vasc Biol. Volume 22, No. 6, June 2002, p. 1002.

In addition to coronary bypass surgery, a current treatment strategy to alleviate vascular occlusion includes percutaneous transluminal coronary angioplasty, expanding the internal lumen of the coronary artery with a balloon. Roughly 800,000 angioplasty procedures are performed in the U.S. each year (Arteriosclerosis, Thrombosis, and Vascular Biology Volume 22, No. 6, June 2002, p. 884). However, 30% to 50% of angioplasty patients soon develop significant restenosis, a narrowing of the artery through migration and growth of smooth muscle cells.

In response to the significant restenosis rate following angioplasty, percutaneously placed endoprostheses have been extensively developed to support the vessel wall and to maintain fluid flow through a diseased coronary artery. Such endoprostheses, or stents, which have been traditionally fabricated using metal alloys, include self-expanding or balloon-expanded devices that are “tracked” through the vasculature and deployed proximate one or more lesions. Stents considerably enhance the long-term benefits of angioplasty, but 10% to 50% of patients receiving stents still develop restenosis. (J Am Coll Cardiol. 2002; 39:183-193. Consequently, a significant portion of the relevant patient population undergoes continued monitoring and, in many cases, additional treatment.

Continued improvements in stent technology aim at producing easily tracked, easily visualized and readily deployed stents, which exhibit the requisite radial strength without sacrificing a small delivery profile and sufficient flexibility to traverse the diseased human vasculature. Further, numerous therapies directed to the cellular mechanisms of accumulation of inflammatory cells, smooth muscle cell proliferation and migration show tremendous promise for the successful long-term treatment of ischemic heart disease. Consequently, advances in coupling delivery of such therapies to the mechanical support of vascular endoprostheses, delivered proximate the site of disease, offer great hope to the numerous individuals suffering heart disease.

While advances in the understanding of ischemic heart disease as a complex chronic inflammatory process take place, traditional diagnostic techniques such as coronary angiography yield to next generation imaging modalities. In fact, coronary angiography may not be at all useful in identifying inflamed atherosclerotic plaques that are prone to producing clinical events. Imaging based upon temperature differences, for example, are undergoing examination for use in detecting coronary disease. Magnetic resonance imaging (MRI) is currently emerging as the state of the art diagnostic for arterial imaging, enhancing the detection, diagnosis and monitoring of the formation of vulnerable plaques. Transluminal intervention guided by MRI is expected to follow. However, metals produce distortion and artifacts in MR images, rendering use of the traditionally metallic stents in coronary, biliary, esophageal, ureteral, and other body lumens incompatible with the use of MRI. Consequently, an emerging clinical need for interventional devices that are compatible with and complementary to new imaging modalities is evident.

In order to address the foregoing needs in the art, much work has been done to develop polymeric endoprostheses that may be erodible. However, there is a need in the art for erodible polymers that exhibit the mechanical properties and performance characteristics required of stents and/or anchors. More specificially, there remains a need for erodible polymers that retain both the elastic modulus and percent elongation to failure that is required for a plastically deformable stent design or anchor design with clinically acceptable elastic recoil and radial strength.

### SUMMARY OF THE INVENTION

A generally tubular polymeric endoprosthesis comprising polymer chains in substantially circumferential orientation is disclosed, such as, for example, wherein more than 25% of the polymer chains in substantially circumferential orientation. The generally tubular polymeric endoprosthesis may comprise a polymer comprising a glass transition temperature greater than 37° C., a percentage strain to yield of 5% or less and a percentage of strain to failure between approximately 30% and 35%. Further, the polymer further comprises a percentage elongation of between approximately 5% and 300%.

A generally tubular polymeric endoprosthesis disclosed herein may further comprise walls comprising an inner diameter and an outer diameter, wherein said walls comprise contours, or variable thickness via said outer diameter. Simiarly, the walls may comprise contours or variable thickness via the inner diameter, or both the inner and outer diameter.

A polymeric endoprosthesis disclosed herein may further comprise a filler material which may be inorganic or organic and may confer radiopacity or enhance visualization under magnetic resonance imaging. The filler material may further improve the elastic modulus of the polymer. Examples of filler material include, but are not limited to, gadolinium, bismuth trioxide, platinum and iridium alloys, and barium sulfate.

A generally tubular polymeric endoprosthesis comprising a ratio of Rₜ/Rₐ of 6 or less, or an average roughness of 0.8 microns or less, or an average roughness of 6 or less as measured on the ISO scale, or an average roughness of 35 microinches or less as measured on the RMS scale is disclosed herein.

A method of manufacturing a generally tubular polymeric endoprosthesis comprises the steps of selecting and heating a polymer; extruding the polymer into a tube; expanding the tube in order to substantially align the polymer chains circumferentially. Additional steps may include cutting the tube according to a desired pattern, and expanding the tube within a mold. The step of expanding the tube may comprise disposing a baffle about one end of the generally tubular endoprosthesis and injecting pressurized air or gas into the generally tubular endoprosthesis, or exposing the generally tubular endoprosthesis to a vacuum pressure.

The method may also comprise the step of annealing the tube, or reducing the surface roughness of the generally tubular polymeric endoprothesis according to a suitable method.

An alternative method of manufacturing a generally tubular polymeric endoprosthesis may comprise the steps of selecting a polymer exhibiting a T_{g} of greater than 37° C. and desired crystallinity; heating the polymer to a temperature above its melting temperature for a predetermined amount of time; cooling the polymer rapidly; heating the material to a temperature within its cold crystallization temperature for a desired period of time; forming a generally tubular endoprosthesis from the polymer; and reducing the surface roughness of the generally tubular endoprosthesis using a suitable method. The suitable method may be selected from the group consisting of heat polishing, solvent polishing and laser polishing. The mold may comprise one or more mold block and one or more mold block insert.

### BRIEF DESCRIPTION OF THE DRAWINGS

FIG. 1 is a graph illustrating the stress-strain curve of a polymer in its natural state in contrast to a polymer processed according to the invention.

FIG. 2 is a graph of the stress-strain curve of a polymer in its natural state.

FIG. 3 is a graph of the stress-strain curves of polymer specimens that have been processed according to one parameter of the invention.

FIG. 4 is a graph of the stress-strain curves of polymer specimens that have been processed according to another parameter of the invention.

FIG. 5 is a graph illustrating differential scanning calorimetry data for pol(L-lactide) (PLLA), illustrating the annealing window according to the invention. FIG. 6 is a schematic illustration of single stream processing according to one parameter of the invention.

FIG. 7 is a schematic illustration of single stream processing according to one parameter of the invention.

FIG. 8 illustrates an end view of alternative die blocks according to the invention.

FIG. 9 illustrates an end view of the die blocks of FIG. 8 in a mated position.

### DETAILED DESCRIPTION OF THE INVENTION

Although the invention herein is not limited as such, some embodiments of the invention comprise materials that are bioerodible. “Erodible” refers to the ability of a material to maintain its structural integrity for a desired period of time, and thereafter gradually undergo any of numerous processes whereby the material substantially loses tensile strength and mass. Examples of such processes comprise hydrolysis, enzymatic and non-enzymatic degradation, oxidation, enzymatically-assisted oxidation, and others, thus including bioresorption, dissolution, and mechanical degradation upon interaction with a physiological environment into components that the patient's tissue can absorb, metabolize, respire, and/or excrete. Polymer chains are cleaved by hydrolysis and are eliminated from the body through the Krebs cycle, primarily as carbon dioxide and in urine. “Erodible” and “degradable” are intended to be used interchangeably herein.

A “self-expanding” endoprosthesis has the ability to revert readily from a reduced profile configuration to a larger profile configuration in the absence of a restraint upon the device that maintains the device in the reduced profile configuration.

“Balloon expandable” refers to a device that comprises a reduced profile configuration and an expanded profile configuration, and undergoes a transition from the reduced configuration to the expanded configuration via the outward radial force of a balloon expanded by any suitable inflation medium.

The term “balloon assisted” refers to a self-expanding device the final deployment of which is facilitated by an expanded balloon.

The term “fiber” refers to any generally elongate member fabricated from any suitable material, whether polymeric, metal or metal alloy, natural or synthetic.

The phrase “points of intersection”, when used in relation to fiber(s), refers to any point at which a portion of a fiber or two or more fibers cross, overlap, wrap, pass tangentially, pass through one another, or come near to or in actual contact with one another.

As used herein, a device is “implanted” if it is placed within the body to remain for any length of time following the conclusion of the procedure to place the device within the body.

The term “diffusion coefficient” refers to the rate by which a substance elutes, or is released either passively or actively from a substrate.

As used herein, the term “braid” refers to any braid or mesh or similar woven structure produced from between 1 and several hundred longitudinal and/or transverse elongate elements woven, braided, knitted, helically wound, or intertwined by any manner, at angles between 0 and 180 degrees and usually between 45 and 105 degrees, depending upon the overall geometry and dimensions desired.

Unless specified, suitable means of attachment may include by thermal melt, chemical bond, adhesive, sintering, welding, or any means known in the art.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric. A polymer is a shape memory polymer if the original shape of the polymer is recovered by heating it above a shape recovering temperature (defined as the transition temperature of a soft segment) even if the original molded shape of the polymer is destroyed mechanically at a lower temperature than the shape recovering temperature, or if the memorized shape is recoverable by application of another stimulus. Such other stimulus may include but is not limited to pH, salinity, hydration, and others.

As used herein, the term “segment” refers to a block or sequence of polymer forming part of the shape memory polymer. The terms hard segment and soft segment are relative terms, relating to the transition temperature of the segments. Generally speaking, hard segments have a higher glass transition temperature than soft segments, but there are exceptions. Natural polymer segments or polymers include but are not limited to proteins such as casein, gelatin, gluten, zein, modified zein, serum albumin, and collagen, and polysaccharides such as alginate, chitin, celluloses, dextrans, pullulane, and polyhyaluronic acid; poly(3-hydroxyalkanoate)s, especially poly(beta-hydroxybutyrate), poly(3-hydroxyoctanoate) and poly(3-hydroxyfatty acids).

Representative natural erodible polymer segments or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Suitable synthetic polymer blocks include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, arboxymethyl cellulose, cellulose triacetate and cellulose sulfate sodium salt. These are collectively referred to herein as “celluloses”.

Examples of synthetic degradable polymer segments or polymers include polyhydroxy acids, polylactides, polyglycolides and copolymers thereof, poly(ethylene terephthalate), poly(hydroxybutyric acid), poly(hydroxyvaleric acid), poly[lactide-co-(epsilon-caprolactone)], poly[glycolide-co-(epsilon-caprolactone)], polycarbonates, poly-(epsilon caprolactone) poly(pseudo amino acids), poly(amino acids), poly(hydroxyalkanoate)s, polyanhydrides, polyortho esters, and blends and copolymers thereof.

The degree of crystallinity of the polymer or polymeric block(s) is between 3 and 80%, more often between 3 and 65%. The tensile modulus of the polymers below the transition temperature is typically between 50 MPa and 2 GPa (gigapascals), whereas the tensile modulus of the polymers above the transition temperature is typically between 1 and 500 MPa.

The melting point and glass transition temperature (T_{g}) of the hard segment are generally at least 10 degrees C., and preferably 20 degrees C., higher than the transition temperature of the soft segment. The transition temperature of the hard segment is preferably between −60 and 270 degrees C., and more often between 30 and 150 degrees C. The ratio by weight of the hard segment to soft segments is between about 5:95 and 95:5, and most often between 20:80 and 80:20. The polymers contain at least one physical crosslink (physical interaction of the hard segment) or contain covalent crosslinks instead of a hard segment. Polymers can also be interpenetrating networks or semi-interpenetrating networks.

Rapidly erodible polymers such as poly(lactide-co-glycolide)s, polyanhydrides, and polyorthoesters, which have carboxylic groups exposed on the external surface as the smooth surface of the polymer erodes, also can be used. In addition, polymers containing labile bonds, such as polyanhydrides and polyesters, are well known for their hydrolytic reactivity. Their hydrolytic degradation rates can generally be altered by simple changes in the polymer backbone and their sequence structure.

Examples of suitable hydrophilic polymers include but are not limited to poly(ethylene oxide), polyvinyl pyrrolidone, polyvinyl alcohol, poly(ethylene glycol), polyacrylamide poly(hydroxy alkyl methacrylates), poly(hydroxy ethyl methacrylate), hydrophilic polyurethanes, HYPAN, oriented HYPAN, poly(hydroxy ethyl acrylate), hydroxy ethyl cellulose, hydroxy propyl cellulose, methoxylated pectin gels, agar, starches, modified starches, alginates, hydroxy ethyl carbohydrates and mixtures and copolymers thereof.

Hydrogels can be formed from polyethylene glycol, polyethylene oxide, polyvinyl alcohol, polyvinyl pyrrolidone, polyacrylates, poly (ethylene terephthalate), poly(vinyl acetate), and copolymers and blends thereof Several polymeric segments, for example, acrylic acid, are elastomeric only when the polymer is hydrated and hydrogels are formed. Other polymeric segments, for example, methacrylic acid, are crystalline and capable of melting even when the polymers are not hydrated. Either type of polymeric block can be used, depending on the desired application and conditions of use.

The use of polymeric materials in the fabrication of endoprostheses confers the advantages of improved flexibility, compliance and conformability, permitting treatment in body lumens not accessible by more conventional endoprostheses.

Fabrication of an endoprosthesis according to the invention allows for the use of different materials in different regions of the prosthesis to achieve different physical properties as desired for a selected region. A material selected for its ability to allow elongation of longitudinal connecting members on the outer radius of a curve in a lumen, and compression on the inner radius of a curve in a vessel allows improved tracking of a device through a diseased lumen. A distinct material may be selected for support elements in order that the support elements exhibit sufficient radial strength. Further, the use of polymeric materials readily allows for the fabrication of endoprostheses comprising transitional end portions with greater compliance than the remainder of the prosthesis, thereby minimizing any compliance mismatch between the endoprosthesis and diseased lumen. Further, a polymeric material can uniformly be processed to fabricate a device exhibiting better overall compliance with a pulsating vessel, which, especially when diseased, typically has irregular and often rigid morphology. Trauma to the vasculature, for example, is thereby minimized, reducing the incidence of restenosis that commonly results from vessel trauma.

An additional advantage of polymers includes the ability to control and modify properties of the polymers through the use of a variety of techniques. According to the invention, optimal ratios of combined polymers, and optimal processing have been found to achieve highly desired properties not typically found in polymers. Regions of higher flexibility and decreased varied hoop strength can be selectively fabricated according to the invention. Trauma to the vasculature, for example, is thereby minimized, reducing the incidence of restenosis that commonly results from vessel trauma.

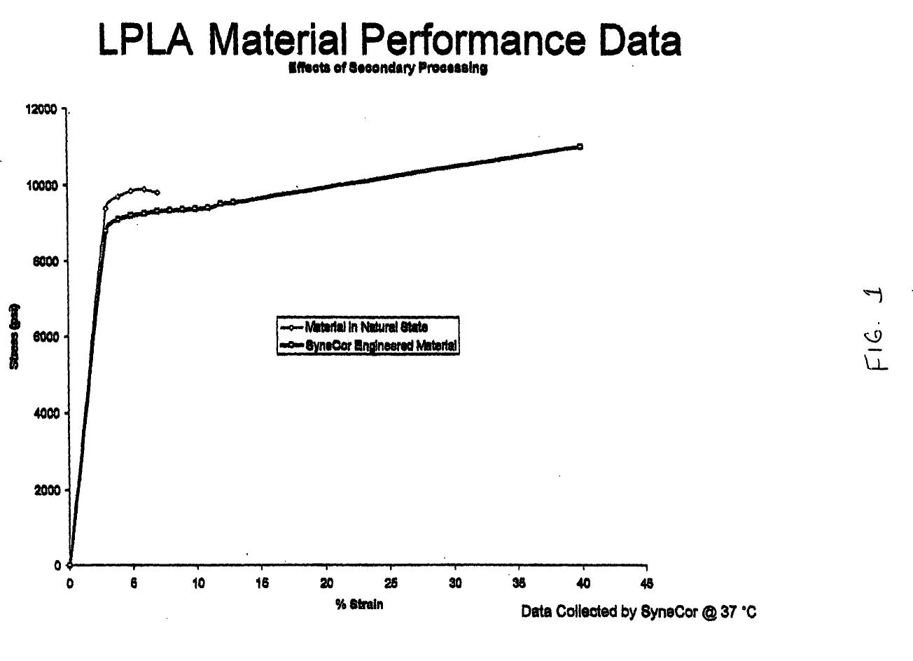

An endoprosthesis manufactured according to the invention has all of the desired properties of polymeric materials, plus increased flexibility and strength as compared to other polymeric endoprostheses. Materials used in the manufacture of endoprostheses must exhibit a glass transition temperature (T_{g}) that is above body temperature. Further, the percentage of strain to yield should be <5%. And the percentage of strain to failure should be 30-35%. Materials processed according to the invention achieve the foregoing requirements. (See FIG. 1.)

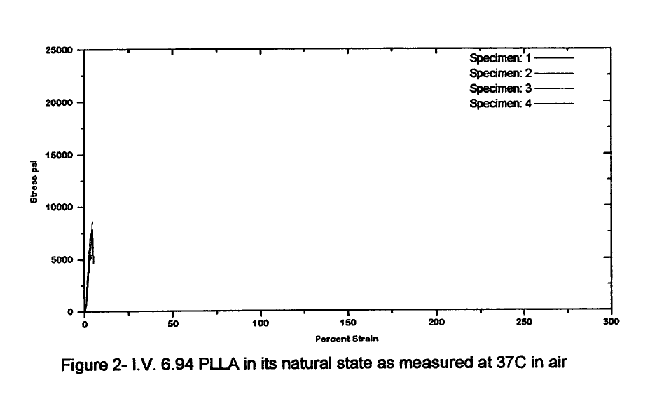

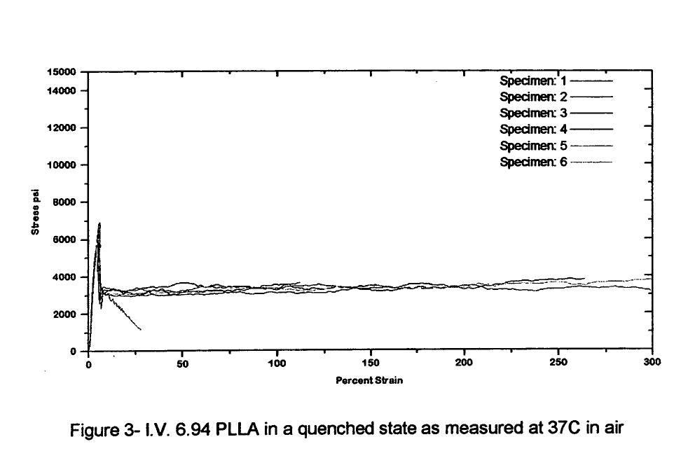

As an example, 100% high molecular weight PLLA is a highly crystalline material that retains the elastic modulus required of a polymeric erodible stent. However, the material in its natural state is too brittle to expand from a rolled down diameter to diameters in the vascular tract. According to the invention, the material may be heated to a temperature above its melting temperature (200° C.-210° C.) for 20-45 seconds (the amount of time and exact temperature are design dependent) and cooled rapidly to quench the material. The foregoing process decreases the percentage of crystallinity, yet has very little effect on the elastic modulus of the material. Further, the percentage elongation may be increased by as much as a factor of 60 (from approximately 5% to as high as 300%). (See FIGS. 2 and 3.)

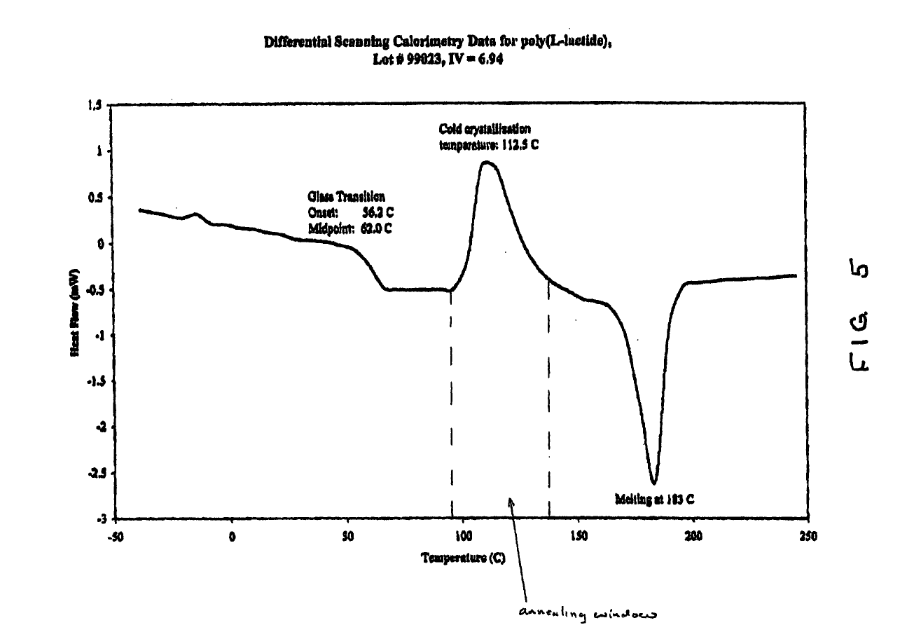

Further, the annealing process (comprising heating the materials according to chosen parameters including time and temperature) increases polymer chain crystallization, thereby increasing the strength of the material. If a more resilient material is added to PLLA in order to increase the % elongation to failure, the resulting material may have a low elastic modulus. Annealing the material will increase the percentage of crystallinity and increase the elastic modulus. By heating the material to a temperature within its cold crystallization temperature (approximately 100° C.-110° C., see FIG. 5) for a period of time that is design and process dependent (10-15 min, for example), the material will have properties that yield acceptable in vitro results. An additional process by which to increase the modulus of elasticity comprises adding biocompatible fillers that may be organic or inorganic, and may include metals. Examples of inorganic fillers include but are not limited to calcium carbonate, sodium chloride, magnesium salts, and others.

An endoprosthesis comprising polymeric materials has the additional advantage of compatibility with magnetic resonance imaging, potentially a long term clinical benefit. Further, if the more conventional diagnostic tools employing angiography continue as the technique of choice for delivery and monitoring, radiopacity can be readily conferred upon polymeric materials. Fillers may be added in order to achieve the foregoing objectives of enhancing radio-opacity and/or enhancing visualization under magnetic resonance imaging. Further examples of fillers that may be suitable to achieve this objective include gadolinium, bismuth trioxide, platinum and iridium alloys, barium sulfate, and others. The foregoing fillers may serve both the purpose of increasing the modulus of elasticity and enhancing the radiopacity and/or visualization under MRI.

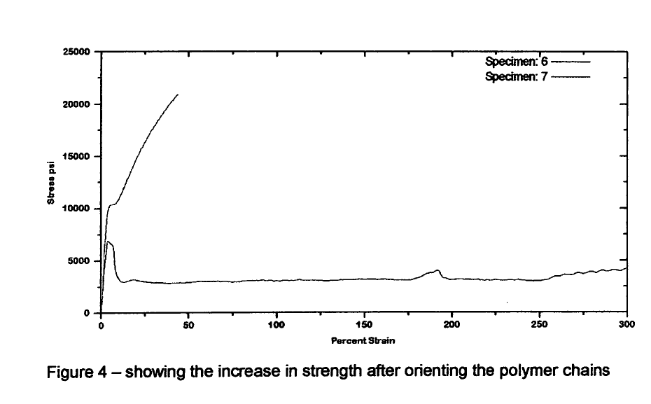

In addition to the annealing process, the polymeric endoprosthesis may be processed to increase the strength of the material. The polymeric chains are generally longitudinally oriented following extrusion. According to the invention, these chains can be substantially reoriented radially, or circumferentially, in order to confer increased hoop strength upon the tubular device. As described in greater detail below, an endoprosthesis such as a stent or an anchor according to the invention may be manufactured according to steps comprising forming a tube from the selected polymers processed as above via an extrusion process and subjecting the tube to gas and pressure within a mold. The step of subjecting the tube to gas and pressure increases the diameter of the tube to a selected diameter and simultaneously aligns the polymeric chains circumferentially. The resulting circumferential orientation of the polymer chains confers increased radial strength upon the finished device. (See FIG. 4.) In addition, the resulting circumferential alignment confers added axial flexibility.

Following trimming to a desired length, the tube may be laser cut according to a design. Then the endoprosthesis may be vapor polished, laser polished, heat polished, or coated to reduce surface imperfections.

Vapor polishing is a surface-smoothing process that is well known in the art to treat polycarbonate, Ultem®, and polysulfone, and also works with PLLA family polymers. The process involves placing the part in a supersaturated environment with a solvent for a controlled period of time until the desired surface finish is achieved. In most cases the solvent will evaporate at or below room temperature but can be heated slightly to accelerate the efficacy of vapor polishing. Care must be taken to prevent erosion of the part itself. The amount of time that the part comes in contact with solvent is design, material and solvent specific. Following the vapor polish process, a heating step may be employed to remove any residual solvents that may reside in the polymer matrix and testing should be done to verify that residual solvents are within acceptable limits. HPLC is one test that can be used to measure solvent levels within a polymer.

According to the invention, it may be possible to simultaneously perform the foregoing heating step and anneal the polymer, if the temperature required in the foregoing heating step is within the cold crystallization range of the polymer. Alternatively, the step of annealing can be performed before, after, or before and after polishing. Further, additional coatings placed on the device for other purposes may provide some added smoothness if the coating integrates itself with the substrate and reduces surface imperfections.

The solvent candidate with the highest vapor pressure is preferred because it will be easier to extract. The following solvents are compatible with PLLA and have the following vapor pressures: Dichloromethane—350 mmHg @ 20° C.; Chloroform—160 mmHg @ 20° C.; Hexafluoroisopropylene—200 mmHg @ 30° C. Additives to the polymeric devices such as drugs or fillers must also be compatible with the selected solvent. In the case of a therapeutic, such as, for example, a pharmaceutical, an incompatible solvent may denature the compound, thereby rendering it ineffective.

Alternatively, the heat polish process is a suitable choice for use with thermoplastic materials. The material is heated to its melting temperature (about 180° C. in the case of PLLA) for a brief period of time until the surface has flowed and the imperfections have been smoothed over. Although this process is effective it must be carefully controlled in order to maintain the desired dimensions of the device geometry. A finished stent can be loaded onto a stainless steel mandrel that rotates at 180 rpm and is inserted into a 180° C. heated tube for 3.5 seconds and then removed. These parameters yield parts with an acceptable surface finish.

As an additional alternative process for smoothing the surface of an endoprosthesis, a process comprises following the laser cutting path with an out of focus pass that will heat the material above melting temperature for the material for a short period of time. This allows the material to momentarily flow and solidify as a smooth surface similar to the above described processes. This process may also be used to reduce surface imperfections as well as create a rounded outer edge of the stent strut which is desirable for atraumatic device trackability. Additionally, the heat affect zone may leave a rib-like contour on the edges adjacent to the laser path which may act as a structural support, thereby imparting additional strength to the device.

The foregoing processes can achieve between 0.2-0.8 microns average roughness (Rₐ). Further, the foregoing processes can achieve a ratio between Rₐ and the total roughness in the test length (Rₜ) of greater than 5. Using the alternative ISO scale of 1-12, 1 being the finest finish, the foregoing processes can achieve 6 or less. And finally, using an RMS scale, a 35 microinches or less can be achieved.

Additionally, the properties of polymers can be enhanced and differentiated by controlling the degree to which the material crystallizes through strain-induced crystallization. Means for imparting strain-induced crystallization are enhanced during deployment of an endoprosthesis according to the invention. Upon expansion of an endoprosthesis according to the invention, focal regions of plastic deformation undergo strain-induced crystallization, further enhancing the desired mechanical properties of the device, such as further increasing radial strength. The strength is optimized when the endoprosthesis is induced to bend preferentially at desired points, and the included angle of the endoprosthesis member is between 40 and 70 degrees.

Curable materials employed in the fabrication of some of the embodiments herein include any material capable of being able to transform from a fluent or soft material to a harder material, by cross-inking, polymerization, or other suitable process. Materials may be cured over time, thermally, chemically, or by exposure to radiation. For those materials that are cured by exposure to radiation, many types of radiation may be used, depending upon the material. Wavelengths in the spectral range of about 100-1300 nm may be used. The material should absorb light within a wavelength range that is not readily absorbed by tissue, blood elements, physiological fluids, or water. Ultraviolet radiation having a wavelength ranging from about 100-400 nm may be used, as well as visible, infrared and thermal radiation. The following materials are examples of curable materials: urethanes, polyurethane oligomer mixtures, acrylate monomers, aliphatic urethane acrylate oligomers, acrylamides, UV polyanhydrides, UV curable epoxies, and other UV curable monomers. Alternatively, the curable material can be a material capable of being chemically cured, such as silicone based compounds which undergo room temperature vulcanization.

Some embodiments according to the invention comprise materials that are cured in a desired pattern Such materials may be cured by any of the foregoing means. Further, for those materials that are photocurable, such a pattern may be created by coating the material in a negative image of the desired pattern with a masking material using standard photoresist technology. Absorption of both direct and incident radiation is thereby prevented in the masked regions, curing the device in the desired pattern. A variety of biocompatibly eroding coating materials may be used, including but not limited to gold, magnesium, aluminum, silver, copper, platinum, inconel, chrome, titanium indium, indium tin oxide. Projection optical photolithography systems that utilize the vacuum ultraviolet wavelengths of light below 240 nm provide benefits in terms of achieving smaller feature dimensions. Such systems that utilize ultraviolet wavelengths in the 193 nm region or 157 nm wavelength region have the potential of improving precision masking devices having smaller feature sizes.

Though not limited thereto, some embodiments according to the invention comprise one or more therapeutic substances that will elute from the surface or the structure or prosthesis independently or as the prosthesis erodes. The cross section of an endoprosthesis member may be modified according to the invention in order to maximize the surface area available for delivery of a therapeutic from the vascular surface of the device. A trapezoidal geometry will yield a 20% increase in surface area over a rectangular geometry of the same cross-sectional area. In addition, the diffusion coefficient and/ or direction of diffusion of various regions of an endoprosthesis, surface, may be varied according to the desired diffusion coefficient of a particular surface. Permeability of the luminal surface, for example, may be minimized, and diffusion from the vascular surface maximized, for example, by altering the degree of crystallinity of the respective surfaces.

According to the invention, such surface treatment and/or incorporation of therapeutic substances may be performed utilizing one or more of numerous processes that utilize carbon dioxide fluid, e.g., carbon dioxide in a liquid or supercritical state. A supercritical fluid is a substance above its critical temperature and critical pressure (or “critical point”). Compressing a gas normally causes a phase separation and the appearance of a separate liquid phase. However, all gases have a critical temperature above which the gas cannot be liquefied by increasing pressure, and a critical pressure or pressure which is necessary to liquefy the gas at the critical temperature. For example, carbon dioxide in its supercritical state exists as a form of matter in which its liquid and gaseous states are indistinguishable from one another. For carbon dioxide, the critical temperature is about 31 degrees C. (88 degrees D) and the critical pressure is about 73 atmospheres or about 1070 psi.

The term “supercritical carbon dioxide” as used herein refers to carbon dioxide at a temperature greater than about 31 degrees C. and a pressure greater than about 1070 psi. Liquid carbon dioxide may be obtained at temperatures of from about −15 degrees C. to about −55 degrees C. and pressures of from about 77 psi to about 335 psi. One or more solvents and blends thereof may optionally be included in the carbon dioxide. Illustrative solvents include, but are not limited to, tetrafluoroisopropanol, chloroform, tetrahydrofuran, cyclohexane, and methylene chloride. Such solvents are typically included in an amount, by weight, of up to about 20%.

In general, carbon dioxide may be used to effectively lower the glass transition temperature of a polymeric material to facilitate the infusion of pharmacological agent(s) into the polymeric material. Such agents include but are not limited to hydrophobic agents, hydrophilic agents and agents in particulate form. For example, following fabrication, an endoprosthesis and a hydrophobic pharmacological agent may be immersed in supercritical carbon dioxide. The supercritical carbon dioxide “plasticizes” the polymeric material, that is, it allows the polymeric material to soften at a lower temperature, and facilitates the infusion of the pharmacological agent into the polymeric endoprosthesis or polymeric coating of a stent at a temperature that is less likely to alter and/or damage the pharmacological agent.

As an additional example, an endoprosthesis and a hydrophilic pharmacological agent can be immersed in water with an overlying carbon dioxide “blanket”. The hydrophilic pharmacological agent enters solution in the water, and the carbon dioxide “plasticizes” the polymeric material, as described above, and thereby facilitates the infusion of the pharmacological agent into a polymeric endoprosthesis or a polymeric coating of an endoprosthesis.

As yet another example, carbon dioxide may be used to “tackify”, or render more fluent and adherent a polymeric endoprosthesis or a polymeric coating on an endoprosthesis to facilitate the application of a pharmacological agent thereto in a dry, micronized form. A membrane-forming polymer, selected for its ability to allow the diffusion of the pharmacological agent therethrough, may then applied in a layer over the endoprosthesis. Following curing by suitable means, a membrane that permits diffision of the pharmacological agent over a predetermined time period forms.

Objectives of therapeutics substances incorporated into materials forming or coating an endoprosthesis according to the invention include reducing the adhesion and aggregation of platelets at the site of arterial injury, block the expression of growth factors and their receptors; develop competitive antagonists of growth factors, interfere with the receptor signaling in the responsive cell, promote an inhibitor of smooth muscle proliferation. Anitplatelets, anticoagulants, antineoplastics, antifibrins, enzymes and enzyme inhibitors, antimitotics, antimetabolites, anti-inflammatories, antithrombins, antiproliferatives, antibiotics, anti-angiogenesis factors, and others may be suitable.

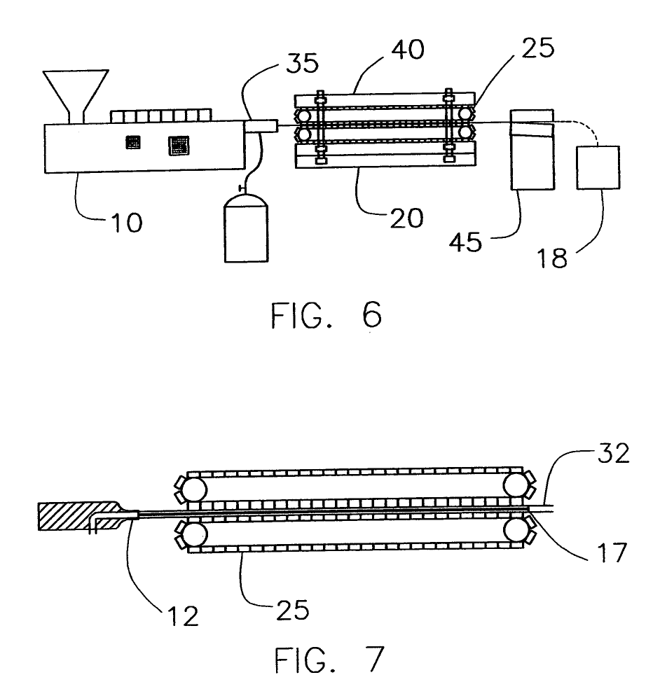

Details of the invention can be better understood from the following descriptions of specific embodiments according to the invention. As an example, in FIGS. 6 and 7, polymer may be synthesized according to desired parameters using desired materials such as those set forth above or as set forth in U.S. patent application Ser. No. 10/342,748 and 10/342,771, which are hereby incorporated in their entirety as if fully set forth herein. Extruded molten tube comprising the foregoing or other suitable polymeric materials from extruder 10 is run over a gas mandrel 12 or baffle assembly of FIG. 7 or directly into a corrugator/blow molder 20 of FIG. 6 where the shape is continuously formed by pressure or vacuum. A continuous loop corrugator tooling track holds matching pairs of molds 25. A typical machine may hold 60-120 pairs of molds. (A typical machine may hold two identical and exact opposite rows of, for example, hardened steel, aluminum, or cast high temperature polymer mold blocks.)

A corrugator may be configured in vertical operation (or over/under) or horizontally where the molds/mold tracks are configured in a side by side configuration The molds are formed/machined in two identical half-rounds which, when positioned opposite each other, form the polymer material into the expanded tubing dimensions. Tubing may be expanded by, for example, between approximately 50% and 80%. More often, an exemplary tube will be expanded by approximately 70% to 75%.

There are three types of forming systems: internal blow molding, vacuum forming, or a combination of the two. Internal blow molding consists of blowing low pressure (0.1-1.5 Bar) through a die-head spider 35 into the center of the continuously extruded hot melt polymer tube (at a temperature depending upon the particular polymer, but in this example within an approximate range of 130°-180° F.). The air is maintained in the tube by a plug or baffle 17 with metallic or silicone washers. The hot melt, under temperature conditions approximately within the range set forth above, is expanded by the internal air pressure against the shape defined by the mold cavity in the machined mold blocks. The blocks may be cooled via cooling plates 40 and thus the material (extrudate 32) is cooled. The extrudate exits the corrugator/blow molder and enters a cutter 45 or spooler (not pictured) and part collection bin 18. The tubing is now ready for secondary annealing or processing such as laser cutting a stent or anchor pattern.

Vacuum forming or molding, most commonly achieved in horizontal machines, consists of pulling the hot melt tubing against the inner diameter of the mold cavity with or by vacuum suction applied through holes in the mold blocks. One advantage of vacuum formed tubing is that it can have various contoured inner diameter walls thicknesses or dimensions. (Both internal blow molding and vacuum forming processes can impart contours to the outer diameter of the extrudate. Contoured surfaces may help impart more strength and rigidity in certain segments and more flexibility in certain other segments of an endoprosthesis.)

Either of these methods will create crystalline orientation in the radial or circumferential bias. Doing so increases the radial strength of tubing which can be directly related to in vivo radial strength increase in vascular scaffolding devices such as stents or in intravascular devices or anchors used to support, hold or stabilize intravascular medical devices. Another advantage of this process is that tubing thickness may be varied. In other words, mold block cavities may be machined with variable surfaces and, in vacuum forming, inner diameter surfaces may be varied as well. Varied surfaces or wall thicknesses may be used to enhance stent or anchor designs by allowing for increased strength or increased flexibility in strategic regions of the device. Variability in wall thickness or surface finish such as, for example, corrugated, ribbed or dimpled (either convex or concave) may allow for increased and strategic drug loading zones and distribution/diffusion points, respectively. A varied inner diameter surface may be used to decrease surface friction on mating devices such as, for example, guide wires. Combined varied surfaces on inner and outer diameter surfaces confer all of the foregoing advantages.

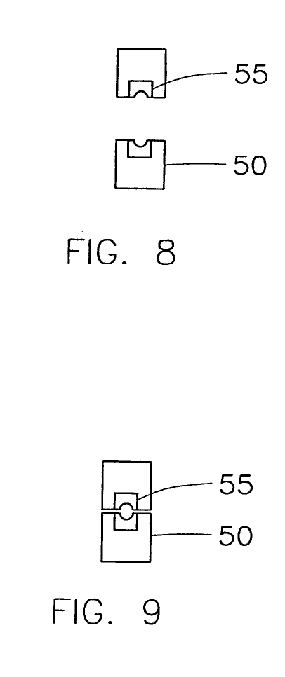

Turning now to FIGS. 8 and 9, alternative mold blocks 50 may comprise aluminum or steel and may further comprise cavity inserts 55 made of phenolic or other high wear, high temperature polymers. Cavity inserts 55 are consequently inexpensive and easily changed tooling parts. Cavity inserts 55 may be held in blocks by recessed socket head cap screw or flat head cap screw. Other suitable materials may be substituted for those listed above.

Alternatively, a single station blow molding may be performed. For example, a preformed short segment of material (or a tubular parison) may be inserted into a cylindrical mold, then heated and expanded under pressure. The polymer of the resulting tubular structure comprises a radial crystalline orientation for improved radial strength.

While particular form of the invention have been illustrated and described above, the foregoing descriptions are intended as examples, and to one skilled in the art will it will be apparent that various modifications can be made without departing from the spirit and scope of the invention

1. A generally tubular polymeric endoprosthesis comprising polymer chains in substantially circumferential orientation.

2. The generally tubular polymeric endoprosthesis according to claim 1 wherein more than 25% of said polymer chains are in substantially circumferential orientation.

3. A generally tubular polymeric endoprosthesis comprising one or more polymers comprising a glass transition temperature greater than 37° C., a percentage strain to yield of 5% or less and a percentage of strain to failure between approximately 30% and 35%.

4. The generally tubular polymeric endoprosthesis according to claim 3 wherein the one or more polymers further comprises a percentage elongation of between approximately 5% and 300%.

5. The generally tubular polymeric endoprosthesis according to claim 1 further comprising walls comprising an inner diameter and an outer diameter, wherein said walls comprise variable thickness via said outer diameter.

6. The generally tubular polymeric endoprosthesis according to claim 1 further comprising walls comprising an inner diameter and an outer diameter, wherein said walls comprise variable thickness via said inner diameter.

7. The generally tubular polymeric endoprosthesis according to claim 1, further comprising walls comprising an inner diameter and an outer diameter, wherein said walls comprise variable thickness along said inner diameter and said outer diameter.

8. The generally tubular polymeric endoprosthesis according to claim 1 further comprising a filler material.

9. The generally tubular polymeric endoprosthesis according to claim 8 wherein said filler material comprises an inorganic material.

10. The generally tubular polymeric endoprosthesis according to claim 9 wherein said filler material confers radiopacity or enhances visualization under magnetic resonance imaging.

11. The generally tubular polymeric endoprosthesis according to claim 9 wherein said filler material confers radiopacity or enhances visualization under magnetic resonance imaging and improves the elastic modulus of the polymer.

12. The generally tubular polymeric endoprosthesis according to claim 9 wherein said filler material is selected from the group consisting of gadolinium, bismuth trioxide, platinum and iridium alloys, and barium sulfate.

13. A generally tubular polymeric endoprosthesis comprising a ratio of Rₜ/Rₐ of 6 or less.

14. A generally tubular polymeric endoprosthesis comprising an average roughness of 0.8 microns or less.

15. A generally tubular polymeric endoprosthesis comprising an average roughness of 6 or less as measured on the ISO scale.

16. A generally tubular polymeric endoprosthesis comprising an average roughness of 35 microinches or less as measured on the RMS scale.

17. A method of manufacturing a generally tubular polymeric endoprosthesis comprising the steps of:
selecting and heating a polymer;
extruding the polymer into a tube;
expanding the tube in order to substantially align the polymer chains circumferentially.

18. The method according to claim 17 with the additional step of cutting the tube according to a desired pattern.

19. The method according to claim 18 wherein the step of expanding the tube comprises expanding the tube within a mold.

20. The method according to claim 17 wherein the step of expanding the tube comprises disposing a baffle about one end of the generally tubular endoprosthesis and injecting pressurized air or gas into the generally tubular endoprosthesis.

21. The method according to claim 17 wherein the step of expanding the tube comprises exposing the generally tubular endoprosthesis to a vacuum pressure.

22. The method according to claim 17 further comprising the step of annealing the tube.

23. The method according to claim 17 with the additional step of:
reducing the surface roughness of the generally tubular polymeric endoprothesis according to a suitable method.

24. The method according to claim 23 wherein the step of smoothing the surface of the generally tubular polymeric endoprosthesis comprises reducing the ratio of Rₜ/Rₐ to 6 or less.

25. A method of manufacturing a generally tubular polymeric endoprosthesis comprising the steps of:
selecting a polymer exhibiting a T_{g} of greater than 37° C. and desired crystallinity;
heating the polymer to a temperature above its melting temperature for a predetermined amount of time;
cooling the polymer rapidly.

26. The method according to claim 25 with the additional step of:
heating the material to a temperature within its cold crystallization temperature for a desired period of time.

27. The method according to claim 26 with the additional steps of:
forming a generally tubular endoprosthesis from the polymer;
reducing the surface roughness of the generally tubular endoprosthesis using a suitable method.

28. The method according to claim 27 wherein the suitable method is selected from the group consisting of heat polishing, solvent polishing and laser polishing.

29. The method according to claim 19 wherein the mold comprises one or more mold block and one or more mold block insert.

30. The generally tubular polymeric endoprosthesis according to claim 1 further comprising walls comprising an inner diameter and an outer diameter, wherein said outer diameter comprises one or more contours.

31. The generally tubular polymeric endoprosthesis according to claim 1 further comprising walls comprising an inner diameter and an outer diameter, wherein said inner diameter comprises one or more contours.

32. The generally tubular polymeric endoprosthesis according to claim 8 wherein said filler material comprises an organic material.

33. The method according to claim 23 wherein the method is selected from the group consisting of heat polishing, solvent polishing and laser polishing.
